(19) United States
(12) Patent Application Publication
(10) Pub. No.: US 2017/0014043 A1
(43) Pub. Date: Jan. 19, 2017

(54) Electrodermal Activity Sensor

(71) Applicant: Galvanic Limited, Dublin (IE)

(72) Inventor: Daragh MCDONNELL, Portmarnick (IE)

(21) Appl. No.: 15/124,372

(22) PCT Filed: Apr. 30, 2014
(86) PCT No.: PCT/EP2014/058881

§ 371 Date: Sep. 8, 2016

(51) Int. Cl. A61B 5/053; C25D 3/38; C23C 18/38; C25D 17/16; C23F 1/18; C25D 5/34; C25D 5/06

(52) U.S. Cl. CPC A61B 5/0531; C25D 5/34; C25D 3/38; C25D 5/06; C25D 17/16; C23F 1/18; C23C 18/38; A61B 2562/125

(57) ABSTRACT

The present invention is directed towards a method of manufacturing a sensor disc for use as a dry electrode in a skin conductance measuring device, the sensor disc comprising a plurality of layers of different materials and the method of manufacturing comprising the steps of etching a copper base layer; electroplating the copper base layer with an intermediate bright copper layer; plating the intermediate bright copper layer with an intermediate palladium plated layer; and, plating the intermediate palladium plated layer with a gold plated surface layer. The advantage of a method of manufacturing a sensor disc in accordance with the present invention is that a roughened surface is created by the etching. This increased roughness corresponds to an increase in surface area of skin in contact with the sensor disc. The larger contact area implies a larger sweat layer between skin and metal, resulting in reduced electrical impedance and hence an improvement in the signal-to-noise ratio of the skin conductance signal detected by the sensor disc. Furthermore, the surface roughness assists in trapping the sweat, also leading to reduced impedance and an improvement in the signal-to-noise ratio of the detected signals. Moreover, in addition to the high performance of the sensor discs manufactured by this process, the sensor discs produced also meet the ergonomic and aesthetic expectations of a contemporary mass market and may be advantageously utilized in a consumer electronics product.

### CROSS REFERENCE TO RELATED APPLICATIONS

This application is a national phase application based upon PCT Application No. PCT/EP2014/058881 filed Apr. 30, 2014 entitled An Electrodermal Activity Sensor, which is hereby incorporated in its entirety herein by reference thereto.

### FEDERALLY SPONSORED RESEARCH AND DEVELOPMENT

None.

### BACKGROUND OF THE INVENTION

1. Field of the Invention

This invention relates to a sensor. In particular, the present invention is directed towards a sensor disc for use as part of a sensor device for measuring electrodermal activity on a user's skin and furthermore to a method for producing such a sensor disc.

2. Background

Throughout this specification, the term “electrodermal activity” shall be understood to encompass any type of activity which results in a change to the electrodermal characteristics of a user's skin.

In recent times, a number of personal electronics products have been put on the market to allow users to monitor their own health characteristics. Such devices generally monitor and measure biometric signals from the user and present these biometric measurements to the user for information and analysis. These devices are typically operational only when in contact with the user and consequently many of the devices are worn continuously or at least used for extended periods of time. Thus, such devices are required to perform robustly outside of a laboratory environment and the devices must also be cost effective and relatively simple to operate.

As a means to obtain the biometric measurements from the user, a plurality of different techniques are employed. Measurement of a user's electrodermal activity, electromyography and electrocardiography all employ electrodes in contact with the skin of a user in order to transduce the corresponding biometric signal.

The performance and reliability of these electrodes is very important in all of the above techniques as the biometric signals obtained by the electrodes are the source input to the devices, which then amplify and process the source input signals so as to deliver the information and results to the user. If the electrodes are poor in initially detecting the biometric signals, then no matter how powerful the amplification and/or processing circuitry, the devices will deliver insufficiently accurate results to the user.

In a laboratory environment or in a clinical setting, silver-silver chloride (Ag—AgCl) electrodes are the preferred type of electrode to be used. These silver-silver chloride electrodes are used in conjunction with a conductive gel which is applied to the skin-engaging surface of the silver-silver chloride electrode in order to reduce the impedance of the electrical path between the user's skin and the skin-engaging surface of the silver-silver chloride electrode. These types of electrodes are colloquially known as “wet electrodes”. These wet electrodes are not suitable for use in the type of personal electronic devices mentioned hereinbefore as the need to apply gel is inconvenient for users. Moreover, it would not be a reasonable expectation of users in everyday, real-world settings to carry with them, and apply periodically, conductive gel to the electrodes of their personal devices. In summary, the use of wet electrodes is not feasible for the types of personal electronic devices which are being brought to market as wearable biometric sensor devices, or even for sensor devices which are not wearable devices, but are envisaged to be used relatively frequently for extended periods of time.

In short, for every day, non-specialist use, an electrode which does not require the application of a conductive gel is desired. Such so-called “dry electrodes” are known from the prior art and are envisaged to be utilized by the personal electronic devices mentioned hereinabove.

Furthermore, as these personal electronic devices will be marketed as consumer products, it is also of importance that the aesthetics of the dry electrodes are appealing to consumers.

The dry electrode of the present invention is designed to be optimized for transduction of electrodermal activity, and specifically for monitoring and measuring skin conductance of a user, as a sign of electrodermal activity. While there are several techniques which are known to be used for the measurement of electrodermal activity, the measurement of skin conductance via application of a constant DC voltage to the skin is believed to be the most widely used. Skin conductance varies widely according to age, sex, race and heredity and also in response to environmental conditions. Skin conductance levels can range from 1 μS to 40 μS dependent on these factors.

Electrodermal activity results from the activity of eccrine sweat glands in a user's body. The sweat produced by these eccrine sweat glands is substantially a solution of sodium chloride, and thusly facilitates the conduction of an electric current. As the activity or inactivity of the eccrine sweat glands will produce more of this sweat or less of this sweat respectively, the activity of the eccrine sweat glands can be quantified by measuring the electrical conductance of the skin. Measuring the skin conductance of the user is accomplished using the dry electrodes to capture and measure the skin conductance biometric signal and then by using associated well-known processing steps, these measured biometric signals, representing the electrodermal activity, are presented to the user.

As the eccrine sweat gland activity is under the control of the sympathetic component of the autonomic nervous system, the eccrine sweat gland activity is an indicator of the activity of the autonomic nervous system. The sympathetic nervous system is a part of an individual's overall nervous system, and the sympathetic component of the autonomic nervous system is the part of the nervous system which mobilizes the individual's so-called “fight-or-flight” response. Consequently, the eccrine sweat gland activity is an indicator of an individual's state of psychological and/or physiological arousal. In this manner, it is possible, using dry electrodes, to measure and quantify a user's psychological and/or physiological arousal.

The greatest density of sweat glands on a human body are to be found on the palmar aspect, namely the anterior side of the hands, and the plantar aspect, namely the soles of the feet. The fingertips contain a relatively high concentration of sweat glands and represent a convenient site for skin conductance measurement, particularly in everyday situations. Therefore, it is common for the dry electrodes to be clamped around a user's fingertips, or to be held in place by a user between their thumb and one of their fingers.

Having selected the fingertips as the most practical and reliable areas on a human's body for measuring skin conductance, it is important to select an appropriate electrode construction to interact with the skin of the fingertips which can be quite ridged compared to other areas of skin on a user. As discussed above, the interaction between the skin on a user's fingertip and the skin-engaging surface of a dry electrode is crucial in obtaining accurate and true skin conductance measurements.

The prior art has considered a large number of different materials for use in dry electrodes, as will be discussed in greater details below, but there are a number of known issues with these electrode designs and manufacturing processes. The problems include a reduction in skin conductance measuring sensitivity over time due to corrosion and other effects which impair the ability of the chosen material to carry out the skin conductance measurement; the cost of the materials; and, the appearance of the materials.

It is a goal of the present invention to provide a method of manufacture of a sensor for use as a dry electrode and a dry electrode apparatus that overcomes at least one of the above mentioned problems in the design and manufacture of dry electrodes.

### SUMMARY OF THE INVENTION

The present invention is directed towards a method of manufacturing a sensor disc for use as a dry electrode in a skin conductance measuring device, the sensor disc comprising a plurality of layers of different materials and the method of manufacturing comprising the steps of etching a copper base layer; electroplating the copper base layer with an intermediate bright copper layer; plating the intermediate bright copper layer with an intermediate palladium plated layer; and plating the intermediate palladium plated layer with a gold plated surface layer.

The advantage of a method of manufacturing a sensor disc in accordance with the present invention is that a roughened surface is created by the etching. This increased roughness corresponds to an increase in surface area of skin in contact with the sensor disc. The larger contact area implies a larger sweat layer between skin and metal, resulting in reduced electrical impedance and hence an improvement in the signal-to-noise ratio of the skin conductance signal detected by the sensor disc.

Furthermore, the surface roughness assists in trapping the sweat, also leading to reduced impedance and an improvement in the signal-to-noise ratio of the detected signals.

Moreover, in addition to the high performance of the sensor discs manufactured by this process, the sensor discs produced also meet the ergonomic and aesthetic expectations of a contemporary mass market and may be advantageously utilized in a consumer electronics product.

In a further embodiment, the method further comprises the step of dipping the sensor disc into a citric acid bath prior to plating the intermediate palladium plated layer with a gold plated surface layer.

In a further embodiment, the method further comprises the step of immersing the copper base layer into a sulphuric acid bath prior to electroplating the copper base layer with the intermediate bright copper layer.

In a further embodiment, the method further comprises the step of degreasing the copper base layer prior to etching the copper base layer.

In a further embodiment, the step of degreasing the copper base layer comprises soak cleaning the copper base layer in an alkaline solution.

In a further embodiment, the step of degreasing the copper base layer comprises performing electrolytic cleaning of the copper base layer in a solution comprising sodium hydroxide, silicon and one or more complexing agents.

In a further embodiment, the step of degreasing the copper base layer comprises initially soak cleaning the copper base layer in an alkaline solution, and subsequently performing electrolytic cleaning of the soaked cleaned copper base layer in a solution comprising sodium hydroxide, silicon and one or more complexing agents.

In a further embodiment, the step of dipping the etched copper base layer in a sulphuric acid dip prior to electroplating the copper base layer with an intermediate bright copper layer.

In a further embodiment, the copper base layer is electroplated with the intermediate bright copper layer which has a thickness in the range of 2 micrometres to 40 micrometres (2 μm→40 μm). In a further embodiment, the copper base layer is electroplated with the intermediate bright copper layer which has a thickness of approximately 10 micrometres (10 μm).

In a further embodiment, the intermediate bright copper layer is plated with an intermediate palladium plated layer which has a thickness in the range of 10 nanometres to 500 nanometres (10 nm→500 nm). In a further embodiment, the intermediate bright copper layer is plated with an intermediate palladium plated layer which has a thickness of approximately 100 nanometres (100 nm).

In a further embodiment, the intermediate palladium plated layer is plated with a gold plated surface layer which has a thickness in the range of 100 nanometres to 10 micrometres (100 nm→10 μm). In a further embodiment, the intermediate palladium plated layer is plated with a gold plated surface layer which has a thickness of approximately 1 micrometre (1 μm).

In a further embodiment, the step of degreasing the copper base layer comprising soak cleaning the copper base layer in an alkaline solution is carried out in a bath having a temperature of approximately 60° C. for approximately 5 minutes.

In a further embodiment, the step of dipping the etched copper base layer in a sulphuric acid dip prior to electroplating the copper base layer with an intermediate bright copper layer is carried out for approximately 120 seconds and is carried out without any agitation.

In a further embodiment, the step of etching the copper base layer is carried out for between 30 seconds and four minutes and is carried out in an etching solution which comprises less than 3 grams of copper per litre of etching solution.

In a further embodiment, the step of etching the copper base layer is carried out for approximately sixty seconds and is carried out in an etching solution which comprises less than 3 grams of copper per litre of etching solution.

The present invention is further directed towards a sensor disc for use as a dry electrode in a skin conductance measuring device, the sensor disc comprising a copper base layer, an intermediate bright copper layer, an intermediate palladium plated layer, and, a gold plated surface layer.

This combination of layers was found to deliver the best trade-off between the criteria of performance, appearance and cost.

In a further embodiment, the intermediate bright copper layer has a thickness in the range of 2 micrometres to 40 micrometres (2 μm→40 μm). In a further embodiment, the intermediate bright copper layer has a thickness of approximately 10 micrometres (10 μm).

In a further embodiment, the intermediate palladium plated layer has a thickness in the range of 10 nanometres to 500 nanometres (10 nm→500 nm). In a further embodiment, the intermediate palladium plated layer has a thickness of approximately 100 nanometres (100 nm).

In a further embodiment, the gold plated surface layer has a thickness in the range of 100 nanometres to 10 micrometres (100 nm→10 μm). In a further embodiment, the gold plated surface layer has a thickness of approximately 1 micrometre (1 μm).

The present invention is further directed towards a sensor disc for use as a dry electrode in a skin conductance measuring device, the sensor disc manufactured according to the process outlined hereinabove.

The present invention is directed to a sensor disc for use as an electrodermal activity measuring electrode, the sensor disc comprising a copper base layer, an intermediate bright copper layer, and intermediate palladium layer and a gold plated surface layer.

This combination of layers was found to deliver the best trade-off between the criteria of performance, appearance and cost.

The process of the present invention is directed towards a process for producing a sensor disc for use as dry electrodes optimized for the transduction of electrodermal activity on the fingertips, and specifically skin conductance. In addition to high performance, the sensor discs thus produced meet the ergonomic and aesthetic expectations of a contemporary mass market and may be utilized in a consumer electronics product.

### DETAILED DESCRIPTION OF THE EMBODIMENTS

The invention will be more clearly understood from the following description of some embodiments thereof, given by way of example only, with reference to the accompanying drawings.

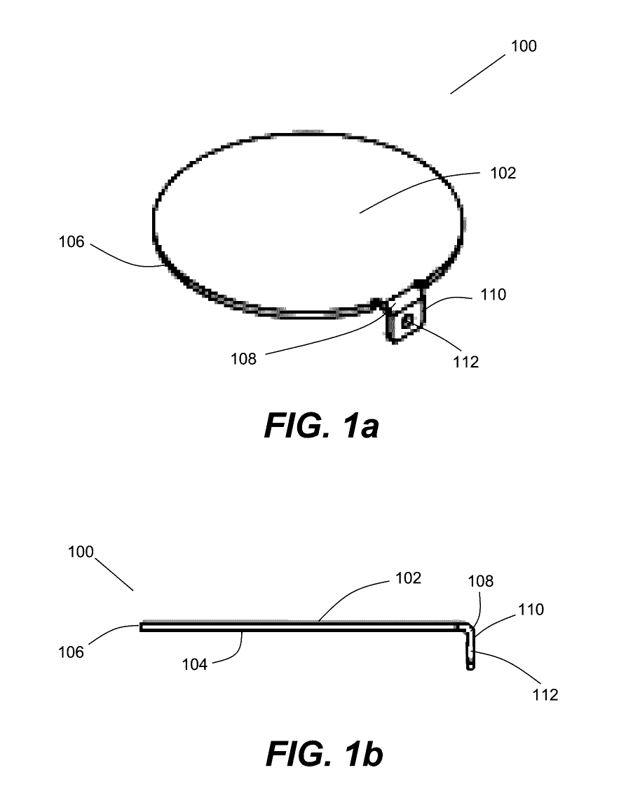

FIG. 1a is a perspective view of a sensor disc in accordance with the present invention.

FIG. 1b is a side elevation view of the sensor disc of FIG. 1a.

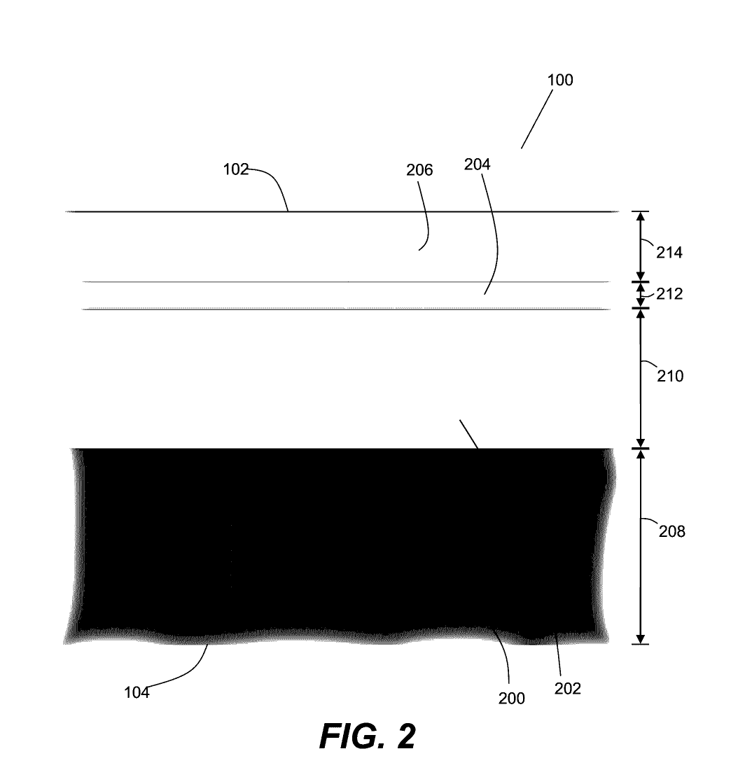

FIG. 2 is a diagrammatic cross-sectional view of the sensor disc of FIG. 1a.

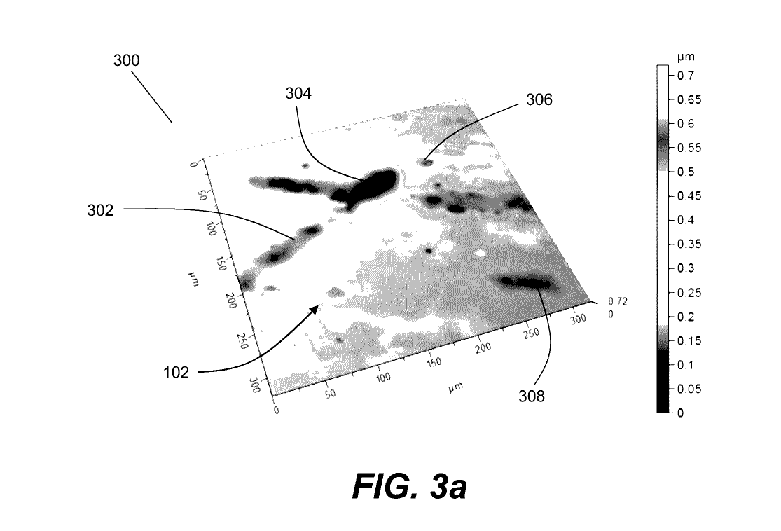

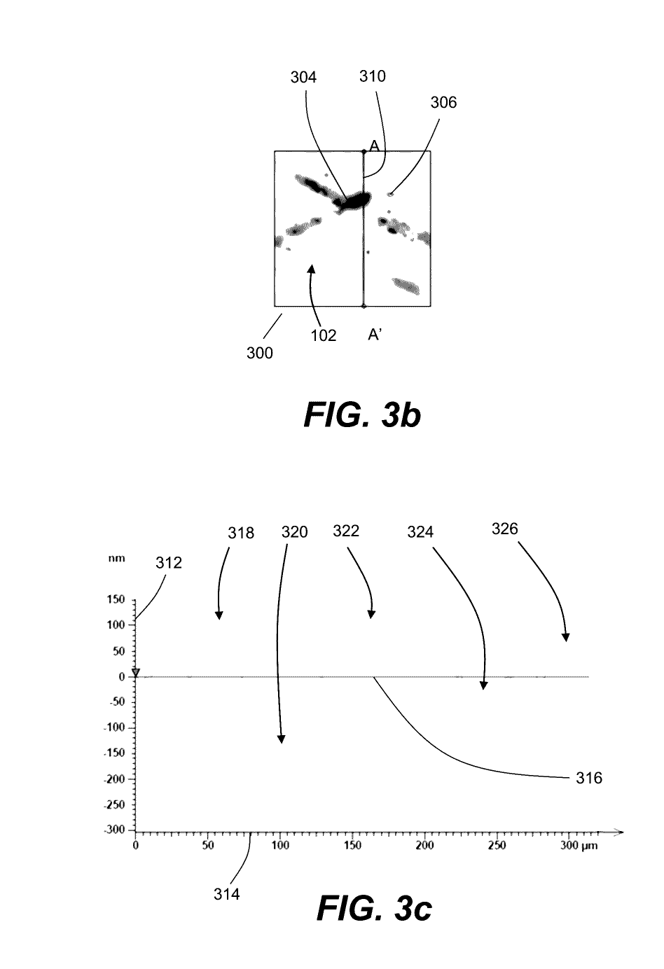

FIG. 3a is a perspective view of a surface topology of a portion of a sensor disc, manufactured in accordance with the present invention, as observed under x-ray fluorescence imaging.

FIG. 3b is a plan view of the surface topology of the portion of the sensor disc of FIG. 3a, as observed under x-ray fluorescence imaging.

FIG. 3c is a graphical representation of the height variance of the portion of the surface topology of the sensor disc of FIG. 3a along a cross-sectional line A-A′.

Referring to FIGS. 1a and 1b, there is provided a sensor disc indicated generally by reference numeral 100. The sensor disc 100 comprises a top face 102, a bottom face 104 and a side wall 106. The top face 102 and bottom face 104 of the sensor disc 100 are substantially circular in shape. A connection lug 110 projects away the sensor disc 100 via a shoulder joint 108. A through hole 112 is arranged on the connection lug 100 and a signal reading made by a top face 102 of the sensor disc 100 is passed through to the connection lug 110 and further on to a wire or a bus (not shown) that may be advantageously connected to the sensor disc 100 by way of the through hole 112. The connecting lug 110 may preferably depend downwardly at a substantially orthogonal angle away from the top face 102 of the sensor disc 100. The wire may be preferably soldered to the sensor disc 100 adjacent the through hole 112 of the connection lug 110 for connection to further electrical signal conditioning and amplification circuitry (not shown).

The sensor disc 100 is envisaged to be used as a dry electrode in a biometric electronics consumer device (not shown)

In the past, as briefly mentioned above, numerous materials have been explored for use as sensor discs for dry electrodes. Typically, these materials have been metals, due to their high electrical conductivity and availability. The table below lists the resistivity, denoted by p, and conductivity, denoted by a, for a number of materials commonly considered for use as sensor discs 100.

TABLE

| | Resistivity | Conductivity |
|---|---|---|
| Material | [ρ (Ω · m × 10⁻⁸) at 20° C.] | [σ (S/m × 10⁷) at 20° C.] |
| Silver | 1.59 | 6.30 |
| Copper | 1.68 | 5.96 |
| Gold | 2.44 | 4.10 |
| Aluminium | 2.82 | 3.5 |
| Platinum | 10.6 | 0.94 |
| Stainless steel | 69.0 | 0.15 |

From the above table, it can be seen that silver, copper and aluminium are attractive candidates as materials for manufacturing the sensor discs 100. Gold is also an attractive candidate however the cost of using gold must be borne in mind. Platinum is similar to gold in terms of the electrical characteristics and costs but has further disadvantages. Stainless Steel is seen to be less attractive to use.

Looking at the most attractive candidates of the various materials in greater detail:
Silver is the most conductive of all the metals. The appearance of silver, particularly when the metal has been polished, is attractive. However, silver is prone to tarnishing in the presence of pollutants such as atmospheric sulphur or hydrogen sulphide, which are plentiful in urban environments. Aesthetically, tarnishing results initially in yellow staining of the silver surface, which can then progress to purple and eventually black discolouration, none of which are attractive from a user's perspective. Moreover, any polishing applied to the silver, which may be considered so as to produce an aesthetically pleasing affect, will have the adverse effect of reducing the surface area of the metal which is in contact with the user's skin. This reduction in surface area will lead to a reduction in sensitivity when measuring the electrodermal activity, through measuring the skin conductance of the user.
Copper is relatively inexpensive, ductile and highly conductive. However, the brown finish of copper does not make it particularly attractive for use in consumer electronics devices. In atmospheric conditions, copper corrodes rapidly producing a blue and/or green patina. This copper oxide substantially reduces surface conductivity.
Aluminium is a good electrical conductor. It spontaneously forms a thin oxide layer that prevents further oxidation, but this layer has a high electrical resistance. Unpolished aluminium has a somewhat dull and unattractive finish. Aluminium can be polished to a mirror finish, but as before, the polishing will reduce the surface area of the aluminium which is in contact with the user's skin, which leads to a reduction in sensitivity when measuring the electrodermal activity of the user using a polished aluminium electrode surface.
Gold is highly conductive and also has an attractive appearance, even when not highly polished. However, it is one of the most expensive precious metals, being only slightly less costly than platinum and considerably more expensive than silver. While gold is relatively expensive to use, gold is very malleable which is an advantage from a manufacturing perspective as the amount of gold to be used can be kept to a minimum by using a thin layer of gold. Gold is highly unreactive and will not form an oxide layer nor corrode at normal air temperatures. Hence it will retain its surface conductivity in everyday use.
Platinum is the least reactive of metals, is highly resistant to corrosion and will not oxidize in air at any temperature. However, its electrical conductivity is significantly less than other precious metals such as gold and silver. It is also extremely rare and thus highly expensive. In unpolished form, platinum comprises a greyish-white colour which is not attractive for consumer products. Polishing the platinum will result in the same sensitivity disadvantages discussed above.

In assessing the various options for materials to use in a sensor disc which is to be utilized as a dry electrode, it became clear that any choice of material would result in a compromise. For aesthetic purposes, which are of utmost importance in respect of an electrode that will be for everyday use by consumers, gold with its attractive appearance even when not highly polished was selected as the material to be used for the skin-engaging surface of the sensor disc 100. As the cost of manufacturing the entire sensor disc 100 from gold would be prohibitive, the present invention adopted a layered design approach.

Therefore, a sensor disc 100 which acts as a dry electrode was designed having a plurality of layers of different materials.

Referring to FIG. 2, the sensor disc 100 comprises a copper base layer 200, an intermediate bright copper layer 202, an intermediate palladium plated layer 204 and a gold plated surface layer 206. The gold plated surface layer 206 forming the skin-engaging surface of the sensor disc 100, which is the top face 102 of the sensor disc 100. A lowermost surface of the copper base layer 200 forms the bottom face 104 of the sensor disc 100. This combination of a copper base layer 200, an intermediate bright copper layer 202, an intermediate palladium plated layer 204 and a gold plated surface layer 206 was deemed to deliver the best combination of electrodermal activity measurement sensitivity, aesthetic appeal and acceptable manufacturing cost.

The thicknesses of the various layers 200, 202, 204 and 206 of materials are also indicated in FIG. 2.

The thickness of the copper base layer 200 is indicated by reference numeral 208. The thickness of the intermediate bright copper layer 202 is indicated by reference numeral 210. The thickness of the intermediate palladium plated layer 204 is indicated by reference numeral 212. And finally, the thickness of the gold plated surface layer 206 is indicated by reference numeral 214.

Preferably, the copper base layer thickness 208 is in the range of 0.2 millimetres (0.2 mm) to 5 millimetres (5 mm), and is advantageously 0.5 millimetres (0.5 mm). Preferably, the intermediate bright copper layer thickness 210 is in the range of 2 micrometres (2 μm) to 40 micrometres (40 μm), and is advantageously 10 micrometres (10 μm). Preferably, the intermediate palladium plated layer thickness 212 is in the range of 10 nanometres (10 nm) to 500 nanometres (500 nm), and is advantageously 100 nanometres (100 nm). Preferably, the gold plated surface layer thickness 214 is in the range of 100 nanometres (100 nm) to 10 micrometres (10 μm), and is advantageously 1 micrometre (1 μm).

The copper base layer 200 is etched in a controlled fashion for a predetermined period to result in a rough surface topology. The copper base layer 200 is then plated with the intermediate bright copper layer 202 which is a layer of bright copper. This intermediate bright copper layer 202 fills out some of the roughness of the etching process; and hence, the intermediate bright copper layer 202 slightly reduces the degree of roughness of surface topology without dispensing with it entirely. This is an important factor in achieving a consistent surface roughness of the sensor disc 100. Additionally, the bright copper of the intermediate bright copper layer 202 helps to brighten the appearance of the sensor disc 100 for a more aesthetically pleasing effect.

The intermediate palladium plated layer 204 is then added. The intermediate palladium plated layer 204 brightens the overall appearance of the sensor disc 100 while also preventing diffusion of the intermediate bright copper layer 202 to the gold plated surface layer 206, which would otherwise cause discolouration of the top face 102 of the sensor disc 100. Palladium is conductive and also exhibits excellent corrosion resistance. Furthermore, the durability of the gold plated surface layer 206 is enhanced by using an under-layer with a hardness value greater than that of gold. The intermediate palladium plated layer 204 has a hardness value which is greater than the hardness value of the gold plated surface layer 206. Therefore, the intermediate palladium plated layer 204 provides increased mechanical support to the sensor disc 100.

Finally, the gold plated surface layer 206 which is in essence a layer of acid hard gold is added to the sensor disc 100 to complete the manufacturing of the sensor disc 100. It should be noted that acid hard gold refers to a gold with a small quantity of added cobalt. When the acid hard gold is used as the gold plated surface layer 206, the durability of the gold plated surface layer 206 is enhanced.

As the gold plated surface layer 206 represents the majority of the costs of the materials which make up the sensor disc 100, preferably only the top face 102 and the connection lug 110 of the sensor disc 100 are plated with the gold plated surface layer 206. There is no substantive loss in performance of the sensor disc 100 as a result of taking this approach. The single-sided plating can be achieved in a number of ways, and a brush plating system for this purpose will be discussed further hereinbelow.

The thicknesses of the gold plated surface layer 206 and the intermediate bright copper layer 202 are important in terms of the manufacture of the sensor disc 100. If either the gold plated surface layer 206 and/or the intermediate bright copper layer 202 is excessively thick, then the surface roughness of the top face 102 of the sensor disc 100, which was introduced by etching of the copper base layer 200, will be smoothened out too much, thus reducing the sensitivity of the sensor disc 100 by reducing the ability of the sensor disc 100 to measure the electrical conductance of the user's skin. The preferred thicknesses mentioned hereinbefore have been found to be most optimal for the sensor disc of the present invention.

Referring now to FIGS. 3a to 3c inclusive, the surface topology 300 of the top face 102 of the sensor disc forms an important part of the overall sensitivity of the sensor disc when used in a dry electrode. As discussed hereinabove, polished surfaces result in reduced sensitivity compared to surfaces with some intentional roughness or unevenness. Polished surfaces result in a bright, reflective, aesthetically-pleasing finish whereas roughened or uneven surfaces disperse the incident light in random directions, producing a dull, matted appearance. There is clearly a trade-off between a rough surface topology 300 on the top face 102 of the sensor disc for sensitivity of measurement of the skin conductance, versus, the aesthetic appearance of the surface finish of the top face 102 of the sensor disc.

FIGS. 3a and 3b show the variation in surface height of a 300 micrometre×300 micrometre (300 μm×300 μm) portion of a top face 102 of a sensor disc in accordance with the present invention. The 300 micrometre×300 micrometre (300 μm×300 μm) portion of the top face 102 of the sensor disc was examined and captured by X-ray fluorescence imaging. This X-ray fluorescence imaging illustrates the variation in surface height with respect to the roughness and unevenness induced to the top face 102 of the sensor disc by the etching process. A variation in surface height of approximately 0.7 micrometres (0.7 μm) is shown in FIGS. 3a and 3b; however, in practice, a surface height variation of between 0.6 micrometres to 1.2 micrometres (0.6 μm→1.2 μm) has been observed. Peaks and troughs 302, 304, 306, 308 indicated on FIGS. 3a and 3b are illustrative of the roughness and unevenness which has been intentionally formed across the portion of top face 102 of the sensor disc.

Referring to FIGS. 3b and 3c, a height profile 315 along a cross-sectional part A-A′ (also indicated by reference numeral 310) of the portion 300 of the top face 102 of the sensor disc of the present invention is shown.

FIG. 3c in particular shows the graphical representation of the variance in surface height along a cross-section 310 of the portion 300 of the sensor disc. This variance in surface topology of the sensor disc is at the crux of the present invention. Peaks and troughs 318, 320, 322, 324, 326 in the surface topology can be seen in FIG. 3c. For example, the trough 304 in FIGS. 3a and 3b is seen as the trough 320 in FIG. 3c. A nominal surface level 316 is also shown in FIG. 3c and the peaks and trough can be determined relative to this nominal surface level 316. The abscissa axis 314 in FIG. 3c denotes the point from 0 to 300 along the 300 micrometre (300 μm) long cross-sectional line 310 shown in FIG. 3b. The ordinate axis 312 of FIG. 3c denotes the height of the surface topology of the cross-section 310 of the portion 300 of the top face 102 of the sensor disc, relative to the nominal surface level 316. The highest peak 318 is approximately 100 nanometres (100 nm) above the surface level 316, and the lowest trough 320 is approximately 215 nanometres (215 nm) below the surface level 316. This results in a variation of approximately 300 nanometres (300 nm) along the cross-section 310 of the portion 300 of top face 102 of the sensor disc.

The intentional roughness and unevenness formed by the etching and subsequent processing manufacture steps results in an increase in an amount of surface area of skin which is held in contact with the top face surface of the sensor disc. This is due to the peaks and troughs causing there to be an increase in surface area on the electrode which the skin of the user can come into contact with. A larger contact area implies a larger sweat layer between skin and metal, resulting in reduced electrical impedance and hence the possibility of increased signal to noise ratio.

Furthermore, the surface roughness assists in trapping sweat between the microscopic peaks and troughs in the roughened and uneven surface of the top face of the sensor disc and this trapped sweat also leads to a reduction in the impedance and consequently an increase in the signal-to-noise ratio of the signals detected by the sensor disc. This increased sensitivity improves the quality of the signal captured at source and provided that the subsequent amplification and processing stages are effected correctly, an accurate skin conductance measurement result should ensue which will lead to an accurate determination of a user's psychological and/or physiological arousal.

A procedure of manufacturing the sensor disc 100 of FIGS. 1a and 1b is now detailed below.

TABLE

| Stage | Description | Parameters | Solution Makeup |
|---|---|---|---|
| Soak Cleaner | Alkaline solution | 5 minutes @ | 30 g/L AK160 |
| | for immersion | 60° C. | |
| | degreasing of | | |
| | copper | | |
| Rinse | | | |
| Electro- | Electrolytic | 8 A/dm² for | 65 g/L EL-DCG |
| cleaner | degreasing of | 2 minutes | |
| | copper | | |
| Rinse | | | |
| Sulphuric | | 2 minutes | 5% v/v |
| Acid | | immersion; | |
| | | no agitation | |
| Rinse | | | |
| Etch | Micro-etching | 1 minute | 75 g/L Slotetch 584, |
| | of copper | immersion; | 10% v/v Sulphuric |
| | | maintain | Acid, 1 g/L copper |
| | | dissolved at | sulphate |
| | | copper < 3 g/L | |
| Sulphuric | | | 5% v/v Sulphuric |
| Acid Dip | | | Acid, 10 seconds |
| Rinse | | | |
| Copper | ~10 micron | 3 A/dm², 20 | Bright copper TB10 |
| Electro- | thickness | minutes, RT | |
| plating | | | |
| Rinse | | | |
| Palladium | Target | 0.5 A/dm² | Makeup as per |
| 2000B | Thickness - | | datasheet, |
| | 0.1 micron | | |
| Rinse | | | |
| Pre-Dip | Adjustment | | 1% v/v Citric Acid |
| Citric Acid | of pH | | |
| Gold Plating | 0.8 to 1.0 | 0.5 A/dm² | Hard Gold Cobalt |
| | micron | | Metgold 2010C VBS |
| | thickness | | Gold Content 4 g/L |
| | | | Platinized Titanium |
| | | | Anodes |
| Rinse | | | |

The above process for preparing and manufacturing a sensor disc comprises sixteen process steps, which are preferable to follow, but it will be readily understood by those skilled in the art that known alternative steps, yielding the same results, may be used in place of the above detailed process steps.

The first step is to soak clean the copper base layer 200 of the sensor disc 100 using an alkaline solution. The copper base layer 200 is immersed in a bath of alkaline solution for approximately five minutes at 60° C. This step is used to degrease the copper base layer 200. An alkaline solution using 30 g/L of a solution comprising, for example sodium hydroxide and phosphate should ideally be used. Such a solution is sold by Dr.-Ing. Max Schlötter GmbH & Co. KG under the product name SLOTOCLEAN AK 160. It will of course be understood that alternative alkaline solutions may be used to degrease the copper base layer 200. In a further embodiment, ultrasound and/or air agitation may be used in conjunction with the alkaline solution to accomplish the step of soak cleaning the copper base layer 200 of the sensor disc 100.

The second step is a rinse step which is carried out on the copper base layer 200 of the sensor disc 100.

The third step is the electrocleaning of the copper base layer 200. This step causes the electrolytic degreasing of the copper base layer 200. Optimally, 6 g/L of a solution comprising sodium hydroxide, silicon and one or more complexing agents such as gluconate is used. Such a solution is sold by Dr.-Ing. Max Schlötter GmbH & Co. KG under the product name SLOTOCLEAN EL DCG. A current density of approximately 8 A/dm2 being applied to the copper base layer 200 for approximately 2 minutes has been found to yield the best results, with the copper base layer 200 receiving cathodic treatment during this electrocleaning step.

The fourth step is to rinse the copper base layer 200.

The fifth step is to immerse the copper base layer 200 in sulphuric acid for 2 minutes without any agitation. The sulphuric acid is made up at a concentration of 5% v/v.

The sixth step is to again rinse the copper base layer 200 of the sensor disc 100.

The seventh step is to etch the copper base layer 200 of the sensor disc 100. As mentioned herein before, the step of etching the copper base layer 200 is a very important step in the manufacture of the sensor disc of the present invention. In order to produce a desired level of surface roughness and unevenness on the copper base layer 200, the copper base layer 200 is etched in a controlled fashion. The duration for which the copper base layer 200 is immersed in the etchant is critical as is the copper content of the etching solution, which increases over an extended period through re-use. Preferably, immersion for 60 seconds at a copper concentration not exceeding 3 g/L is carried out. This was found to produce etched copper base layers 200 that performed consistently well. However, it will be appreciated that different immersion times may be used provided that the immersion time used results in a sufficient amount of etching on the surface such as to create the desired degree of unevenness and roughness. The immersion is thusly envisaged to be carried out for any period within the range of 30 seconds to 240 seconds, at a copper concentration not exceeding 3 g/L is carried out. If the copper concentration exceeded this value, sensitivity was found to drop off significantly. Etching of the copper base layer 200 provides a consistent baseline for the subsequent plating process to be applied as the etching compensates for variations in surface roughness of the untreated copper base layer. The etching solution is made up of 75 g/L of a first solution comprising a non-persulphate salt-based microetch; such a first solution is sold by Dr.-Ing. Max Schlötter GmbH & Co. KG under the product name SLOTETCH 584. Furthermore, the etching solution is additionally made up of 10% v/v Sulphuric Acid and 1 g/L of copper sulphate.

In the eighth step, the etched copper base layer 200 of the sensor disc 100 is dipped into a sulphuric acid dip, which has a composition make-up of 5% v/v Sulphuric Acid. The etched copper base layer 200 is dipped for approximately 10 seconds.

The ninth step is a further rinsing step.

The tenth step in the process is the step of copper electroplating the copper base layer 200 with the intermediate bright copper layer 202. The intermediate bright copper layer 202 is plated to the copper base layer 200 preferably at a thickness of approximately 10 micrometres (10 μm). A bright copper solution for plating the copper base layer 200 is preferably used. Such a bright copper is sold by Dr.-Ing. Max Schlötter GmbH & Co. KG under the product name BRIGHT COPPER TB 10. The bright copper is electroplated to the copper base layer 200 using a current density of approximately 3 A/dm² for a period of 20 minutes at room temperature.

The next and eleventh step in the process is to again rinse the copper base layer 200 which has been electroplated with the intermediate bright copper layer 202.

The twelfth step is to plate the intermediate bright copper layer 202 with the intermediate palladium plated layer 204. The intermediate palladium plated layer 204 may be preferably formed by using PALADIUM 2000B. The intermediate palladium plated layer 204 is plated to a thickness of approximately 100 nanometres (100 nm). A current density of approximately 0.5 A/dm² is preferably used.

The thirteenth step is to again rinse the copper base layer 200 which has now been electroplated with the intermediate bright copper layer 202 and the intermediate palladium plated layer 204.

The fourteenth step is a pH adjustment step. The copper base layer 200 which has now been electroplated with the intermediate bright copper layer 202 and the intermediate palladium plated layer 204 is briefly dipped into citric acid, which is preferably at a volume-volume concentration of 1% v/v. This will prepare the copper base layer 200 which has now been electroplated with the intermediate bright copper layer 202 and the intermediate palladium plated layer 204 to receive the gold plated surface layer 206.

The fifteenth step is to plate the sensor disc 100 with its gold plated surface layer 206 which will become the skin-engaging surface of the sensor disc 100. The gold plated surface layer 206 is made up to a thickness in the range of 0.8 micrometres to 1 micrometres (0.8 μm→1 μm). The acid hard gold used for forming the gold plated surface layer 206 will be a cobalt-enriched gold such as that sold by Metalor Technologies (UK) Limited under the product name METGOLD 2010C (VBS). A current density of 0.5 A/dm² has been found to be particularly effective during the plating process and platinized titanium anodes are advantageously used. A gold content of approximately 4 g/L has been found to yield the best results. As noted previously, the price of gold dominates the material cost of the sensor disc 100; therefore a significant cost saving can be achieved by plating just the top face 102 of the sensor disc 100, which is the skin-engaging surface of the sensor disc 100. In a further embodiment, the top face 102 of the sensor disc 100, which is the skin-engaging surface of the sensor disc 100, is plated in addition to the connection lug 110 which is also plated with the gold plated surface layer 206 so that there is continuity of the gold plated surface layer 206 all the way to the through hole 112 of the connection lug 110 for connection to the further electrical signal conditioning and amplification circuitry by way of a wired connection. One possible approach is to use a high melting point, “stopping-off” wax. Numerous stop-off approaches are possible, including removal of wax from selected areas, or, lacquers and films to prevent wax from initially adhering to selected areas, and so on.

An alternative technique to the stopping-off technique is the brush plating technique.

This brush plating technique will allow selective plating of the sensor disc's 100 surfaces by use of a brush. The brush is typically made of stainless steel wrapped in an absorbent material such as polypropylene wool. The wrapping material absorbs the plating solution for forming the gold plated surface layer 206. The sensor disc 100 is connected to the cathode of a DC power source and the brush is connected to the anode. As the brush moves over the sensor disc 100, a gold plating is deposited on the surface beneath the brush, which would be the top face 102 of the sensor disc 100 and the connection lug 110 of the sensor disc 100 in accordance with a preferred embodiment of the present invention.

A plating assembly for brush plating batches of sensor discs 100 may be used to speed up this step in the process and overcome the perceived inefficiencies of using brush plating for mass production. The batches of sensor discs 100 would be placed in a vacuum deck specifically constructed to comprise a plurality of receiving slots to accommodate the form factor of a plurality of the sensor discs 100 and the vacuum deck would be fitted with a bus arrangement of cathodes that make contact with the underside of the sensor discs 100 to be plated when the sensor discs 100 are seated in the receiving slots on the deck. Within the plating assembly, the brush would be transported over the top faces 102 of the sensor discs 100 by means of a carriage mounting. The motion of the carriage mounting could be controlled manually, or preferably automated by means of computer control. Plating solution for the gold plated surface layer 206 is supplied continuously to the brush via a transfer pump, which can also be automatically controlled to deliver the plating solution at the desired rate. This brush plating method step is seen to be highly effective and efficient in comparison to known techniques for plating sensor discs 100.

The sixteenth and final step of the process is to rinse the manufactured sensor disc 100 so as to prepare the sensor disc 100 for installation in an electronics device and use as a dry electrode for measuring the electrodermal activity of a user, by measuring the skin conductance of the user.

While the sensor disc 100 has been described as a disc throughout the preceding specification, and has been further referred to having a substantially circular form factor, the person skilled in the art would understand that any number of different form factors which are not disc-like or circular may be used.

Moreover, while specific products from specific manufacturers have been referred to in the manufacturing process, it will be of course understood that alternative products offering the same desired effects may be used in place of the specifically mentioned products.

The term “sensor disc” used throughout the preceding specification, may refer to the fully manufactured sensor disc comprising all of the layers of different manufacturing materials, and/or, to a partially manufactured sensor disc comprising one or more of the different manufacturing materials.

The terms “comprise” and “include”, and any variations thereof required for grammatical reasons, are to be considered as interchangeable and accorded the widest possible interpretation.

It will be understood that the components shown in any of the drawings are not necessarily drawn to scale, and, like parts shown in several drawings are designated the same reference numerals.

The invention is not limited to the embodiments hereinbefore described which may be varied in both construction and detail.

1. A method of manufacturing a sensor disc for use as a dry electrode in a skin conductance measuring device, the sensor disc comprising a plurality of layers of different materials and the method of manufacturing comprising the steps of:
etching a copper base layer;
electroplating the copper base layer with an intermediate bright copper layer;
plating the intermediate bright copper layer with an intermediate palladium plated layer; and
plating the intermediate palladium plated layer with a gold plated surface layer.

2. A method of manufacturing a sensor disc as claimed in claim 1, wherein the method further comprises the step of dipping the sensor disc into a citric acid bath prior to plating the intermediate palladium plated layer with a gold plated surface layer.

3. A method of manufacturing a sensor disc as claimed in claim 1, wherein the method further comprises the step of immersing the copper base layer into a sulphuric acid bath prior to electroplating the copper base layer with the intermediate bright copper layer.

4. A method of manufacturing a sensor disc as claimed in claim 1, wherein the method further comprises the step of degreasing the copper base layer prior to etching the copper base layer.

5. A method of manufacturing a sensor disc as claimed in claim 4, wherein the step of degreasing the copper base layer comprises soak cleaning the copper base layer in an alkaline solution.

6. A method of manufacturing a sensor disc as claimed in claim 4, wherein the step of degreasing the copper base layer comprises performing electrolytic cleaning of the copper base layer in a solution comprising sodium hydroxide, silicon and one or more complexing agents.

7. A method of manufacturing a sensor disc as claimed in claim 4, wherein the step of degreasing the copper base layer comprises initially soak cleaning the copper base layer in an alkaline solution, and subsequently performing electrolytic cleaning of the soaked cleaned copper base layer in a solution comprising sodium hydroxide, silicon and one or more complexing agents.

8. A method of manufacturing a sensor disc as claimed in claim 1, wherein the step of dipping the etched copper base layer in a sulphuric acid dip prior to electroplating the copper base layer with an intermediate bright copper layer.

9. A method of manufacturing a sensor disc as claimed in claim 1, wherein the copper base layer is electroplated with the intermediate bright copper layer which has a thickness in the range of 2 micrometres to 40 micrometres (2 μm→40 μm).

10. (canceled)

11. A method of manufacturing a sensor disc as claimed in claim 1, wherein the intermediate bright copper layer is plated with an intermediate palladium plated layer which has a thickness in the range of 10 nanometres to 500 nanometres (10 nm→500 nm).

12. (canceled)

13. A method of manufacturing a sensor disc as claimed in claim 1, wherein the intermediate palladium plated layer is plated with a gold plated surface layer which has a thickness in the range of 100 nanometres to 10 micrometres (100 nm→10 μm).

14. (canceled)

15. A method of manufacturing a sensor disc as claimed in claim 5, wherein the step of degreasing the copper base layer comprising soak cleaning the copper base layer in an alkaline solution is carried out in a bath having a temperature of approximately 60° C. for approximately 5 minutes.

16. A method of manufacturing a sensor disc as claimed in claim 8, wherein the step of dipping the etched copper base layer in a sulphuric acid dip prior to electroplating the copper base layer with an intermediate bright copper layer is carried out for approximately 120 seconds and is carried out without any agitation.

17. A method of manufacturing a sensor disc as claimed in claim 1, wherein the step of etching the copper base layer is carried out for one minute and is carried out in an etching solution which comprises less than 3 grams of copper per litre of etching solution.

18. A sensor disc for use as a dry electrode in a skin conductance measuring device, the sensor disc comprising:
a copper base layer;
an intermediate bright copper layer;
an intermediate palladium plated layer; and
a gold plated surface layer.

19. A sensor disc as claimed in claim 18, wherein the intermediate bright copper layer having a thickness in the range of 2 micrometres to 40 micrometres (2 μm→40 μm).

20. (canceled)

21. A sensor disc as claimed in claim 18, wherein the intermediate palladium plated layer having a thickness in the range of 10 nanometres to 500 nanometres (10 nm→500 nm).

22. A sensor disc as claimed in claim 18, wherein the intermediate palladium plated layer having a thickness of approximately 100 nanometres (100 nm).

23. A sensor disc as claimed in claim 18, wherein the gold plated surface layer having a thickness in the range of 100 nanometres to 10 micrometres (100 nm→10 μm).

24. (canceled)

25. A sensor disc for use as a dry electrode in a skin conductance measuring device, the sensor disc manufactured according to the method of claim 1.
